(19) United States
(12) Patent Application Publication
(10) Pub. No.: US 2025/0235302 A1
(43) Pub. Date: Jul. 24, 2025

(54) Urinary Incontinence Threshold Device

(71) Applicants: Douglas Larson, Ann Arbor, MI (US); Thomas Danowski, Sycamore, IL (US)

(72) Inventors: Douglas Larson, Ann Arbor, MI (US); Thomas Danowski, Sycamore, IL (US)

(21) Appl. No.: 19/022,238

(22) Filed: Jan. 15, 2025

Related U.S. Application Data
(60) Provisional application No. 63/623,737, filed on Jan. 22, 2024.

(51) Int. Cl. A61F 2/00

(52) U.S. Cl. CPC A61F 2/0054; A61F 2/0009

(57) ABSTRACT

The present invention relates to a Urinary Incontinence Threshold Device (UITD) which encircles and applies pressure to a penis to stop urine flow. This is accomplished by an upper and lower member held together by O-rings on each side of the device. Pressure is applied to the penis by a disc attached to a threaded rod that passes through an adjusting nut located in the center of the upper member. By turning the adjusting nut, the pressure disc is raised or lowered changing the pressure being applied. Catches and latches attached to each side of the UITD allow the unit to be in a hold-open position for ease of installation. After installation, the latches are released by sequentially applying a diagonal force to opposing corners of the UITD. Scales located on each side of the UITD indicate the amount of pressure being applied to the penis.

### RELATED APPLICATIONS

This application claims the benefit of U.S. Provisional Patent Application No. 63/623,737 filed on Jan. 22, 2024, which is hereby incorporated by reference in its entirety.

### FIELD OF THE INVENTION

The present disclosure relates to a device for controlling or mitigating urinary leakage associated with male urinary incontinence, methods for controlling the same, methods for using the device and methods for installing the device.

### BACKGROUND OF THE INVENTION

Male incontinence results from surgeries and radiation treatments that impact the bladder, prostate, and urinary tract as well as a result of the natural aging process. Incontinence can be addressed with several products from absorbent pads/underwear as well as mechanical devices such as urinary incontinence devices/clamps and Artificial Urinary Sphincters (AUS). The problem with incontinence pads and underwear is not just inconvenience and cost but their contribution to waste disposal. AUS devices require a surgical procedure to install which can cost 10's of thousands of dollars to install as well as repair or remove when their useful life has been realized. Moreover, not all patients are suitable for surgical treatment. Thus, urinary incontinence clamp devices have been an acceptable approach in recent years because of lower cost as well as ease of use to treat male incontinence. Urinary incontinence clamps clamp the penis in such a way as to compress or collapse the urethra to prevent or reduce urinary leakage. However, there are some serious drawbacks to the currently available male incontinence clamp devices. For example, these clamping devices typically have limited adjustability and are hinged on one side which results in nonuniform loading on the penis which causes instability and discomfort. The end-user must also remove the device to urinate. Also, because urinary incontinence clamps devices clamp across the top and underside of the penis, they affect blood flow in the penis which can be very uncomfortable and possibly cause damage to the penis especially if installed too tightly or worn too long. Therefore, if used for extended periods of time the clamp may cause serious complications, including pain, urethral erosion, obstruction, and edema. In fact, there was a report of urethral diverticulum in a patient using a penile compression device (Kalra, S. et. al., BMJ, Case Reports 2015. doi; 10.1136/bcr-2015-209957) Thus, it is recommended that these devices be removed/unclamped at a maximum every 4 hours. These clamping devices also utilize cushioning material to help minimize discomfort. However, cushioning material is subject to taking a set as well as harboring microorganisms, such as, bacteria. Finally, clamp devices have a tendency to slide off the end of the penis. The use of cushioning material with recess patterns that increase the friction between the cushioning material and the skin of the penis doesn't solve the problem because the problem is the “liver-like” interior penis material that allows the clamp and skin to slide relative to the urethra. Thus, there remains a need to develop an incontinence device for controlling or mitigating urinary leakage associated with urinary incontinence in males without the undesirable properties of the prior art devices. We have found that the urinary incontinence threshold device (UITD) of the present invention overcomes the drawbacks and undesirable properties of the currently used devices. The present invention relates to a device that simulates what any male has or can easily experience, that full urinary flow during urination can be stopped by simply holding the top and underside of the penis between the thumb and forefinger and applying a relatively light pressure. The UITD utilizes an adjustable pressure disc that simulates the function of the thumb restrained by the structure of the UITD that simulates the forefinger. The infinitely adjustable screw device allows the user to apply just the right amount of gentle vertical pressure to control urinary flow. The UITD adjustment system ensures a personalized fit, allowing users to customize the level of pressure according to their individual needs. The UITD has a scale from 1 to 6 at each side of the device to measure the actual pressure being applied to the penis. The pressure level applied by the UITD needs only be just enough to stop the urine leakage, allowing the user to push urine through the UITD when the user needs to urinate. The UITD also includes an easily removed and installed insert to accommodate smaller girth penises. Moreover, the ergonomic design and materials of construction along with a “Hold Open” feature that allows for ease of installation ensures ease of use, and maximum comfort, enabling individuals to wear the device throughout the day without discomfort or irritation.

### SUMMARY OF THE INVENTION

The present device is directed to an infinitely adjustable screw device attached to a pressure disc that is coupled to parallel springy members to externally apply well defined vertical pressure to control urinary flow through the penis. This pressure is transmitted to the urethra and functions like a pressure-relief valve that allows the flow of urine to be stopped (threshold of urine stoppage) at a given pressure yet allows the user to push urine through the pressure-relief valved area of the penis when desired. The applied pressure of this system operates below the normal blood pressure levels in the penis thus eliminating the issues with current urinary incontinence clamps that reduce or stop blood flow in the penis and must be removed for the user to urinate and are recommended to be removed every two and not longer than four hours.

Accordingly, one exemplary embodiment of the present invention is directed to a male urinary incontinence threshold device comprising:
an upper member;
a lower member having a curved surface wherein the upper and lower members are dimensioned to enclose a penis of a user;
vertical guides attached to the upper member which allow the upper and lower member to be oriented with respect to each other;
channels located in the lower member which engage the vertical guides and allow the upper and lower members to move up and down vertically parallel to each other;
O-rings at each end which elastically hold the upper and lower members together;
O-ring groves located in the upper and lower members to hold the O-rings in place;
a catch on the right and left sides of the lower member;
a channel in the upper member to allow the catches to slide vertically;
a latch in the upper member which engages with the ends of the right and left catches to maintain the upper and lower members in a hold-open position during installation;
diagonal corners located in the upper and lower members which when diagonally squeezed in sequence allow the left and right latches to be disengaged from the catches;
a pressure disc;
a threaded rod in which the pressure disc is molded onto the end of the threaded rod;
an adjustment nut which is positioned in and rotates in the upper member and wherein the threaded rod is threaded through;
an O-ring which is stretched over an O-ring grove in the adjustment nut to hold the adjustment nut in place with respect to the upper member;
a button snapped onto the upper end of the threaded rod which prevents the button from separating from the threaded rod and helps pull the upper and lower members apart;
the adjustment nut is turned by means of convolutions around the circumference of the adjustment nut such that when turned clockwise relative to the button, the pressure is increased; and
a scale molded onto both the left and right vertical outside surfaces of the upper member which indicates the amount of pressure that the disc is applying to the penis.

Another embodiment of the present invention is directed to a method of controlling or mitigating urinary leakage associated with urinary incontinence and stopping the flow of urine in a male as needed comprising use of the previous device.

A further embodiment of the present invention is directed to a method of installing the device onto the penis to ensure proper stability.

An additional embodiment of the present invention is directed to the addition of an easily installed insert to accommodate smaller girth penises.

This allows the pressure disc and UITD structure to trap the “liver-like” interior penis material and prevent the UITD from slipping off the end of the penis due to the length and or amount of the penis material being pressurized by the pressure disc.

### BRIEF DESCRIPTION OF THE DRAWINGS

The drawings are set forth to illustrate particular embodiments and aspects of the invention and should not be construed as limiting the full scope as defined by the appended claims. Thus, the various features, advantages and other uses of the present device will become more apparent and better understood by referring to the following detailed description and drawings in which:

FIG. 1 is an isometric view of the device in the closed position.

FIG. 2 is an isometric view of the device in the hold-open position.

FIG. 3 is a side view of the device in the closed position.

FIG. 4 is a side view of the device in the nominal installed position.

FIG. 5 is a front cross-section view of the device in the nominal installed position.

FIG. 6 is a comparison of why the UITD offers increased resistance to sliding off the end of the penis.

FIG. 7 is a graph of the applied pressure in mm of Hg as a function of the gap.

### DETAILED DESCRIPTION OF THE INVENTION

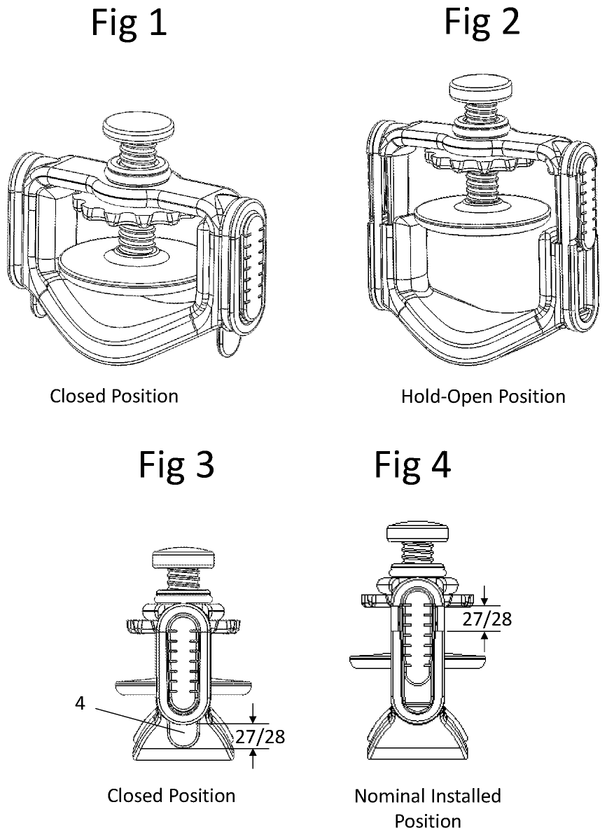

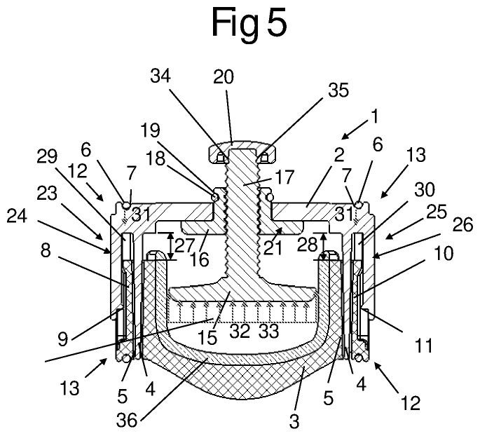

Referring to FIGS. 1 through 5 shows one embodiment of the present invention that includes a UITD 1 which comprises an upper member 2 and a lower member 3 oriented with respect to each other by guide 4 attached to the upper member 2 that engages with channels 5 located in the lower member 3 that allows the upper member 2 and lower member 3 to only move up and down vertically parallel to each other. The upper member 2 and lower member 3 are elastically held together by means of O-ring 6 located in O-ring groove 7. Catch 8 attached to the lower member 3 slides in a channel 29 in the upper member 2. Catch 10 attached to the lower member 3 slides in a channel 30 in the upper member 2. When separated to the hold-open position the end of catch 8 engages with latch 9 in the upper member 2, and the end of catch 10 engages with latch 11 in the upper member 2 to maintain the upper member 2 and lower member 3 in a parallel hold-open position. Catch 8 is disengaged from latch 9 by diagonally squeezing diagonal corners 13, and catch 10 is disengaged from latch 11 by diagonally squeezing diagonal corners 12. The heart of the UITD 1 is the pressure disc 15 that is molded onto the end of the threaded rod 17 that is threaded through the adjustment nut 16 that rotates in the upper member 2 and held in place by O-ring 18 that is stretched over O-ring groove 19 in adjustment nut 16. The upper member 2 and lower member 3 are pulled apart by means of the button 20 snapped onto the end of threaded rod 17, and the lower member 3. The groove 35 in the button 20 engages the projection 34 on the threaded rod 17 to prevent the button 20 from detaching from the threaded rod 17. The scale 24 is molded into the outside surface 23 of the upper member 2 and the scale 26 is molded into the outside surface 25 of the upper member 2 that indicates how far the gap 27 and gap 28 are open, respectively. The distance between the markings on the scale 24 and scale 26 is 0.100 inches. The pressure 32 being applied to the penis is equal to the sum of the forces 31 generated by the O-rings 6 divided by the projected area 33 of the pressure disc 15. The force 31 is directly proportional to the gaps 27 and 28.

As a result of the unique and novel design the UITD 1 of the present invention has among others the following advantages properties, such as, for example, one size fits all; pressure control; and stability.

ONE SIZE FITS ALL—Unlike currently available male incontinence devices, as a result of the threaded rod 17 and the adjustment nut 16 the opening in the device can be made as large as needed to accommodate the full range of penis sizes. And the use of the easily added and removed insert 36 to the lower member 3 accommodates large differences in penis girth.

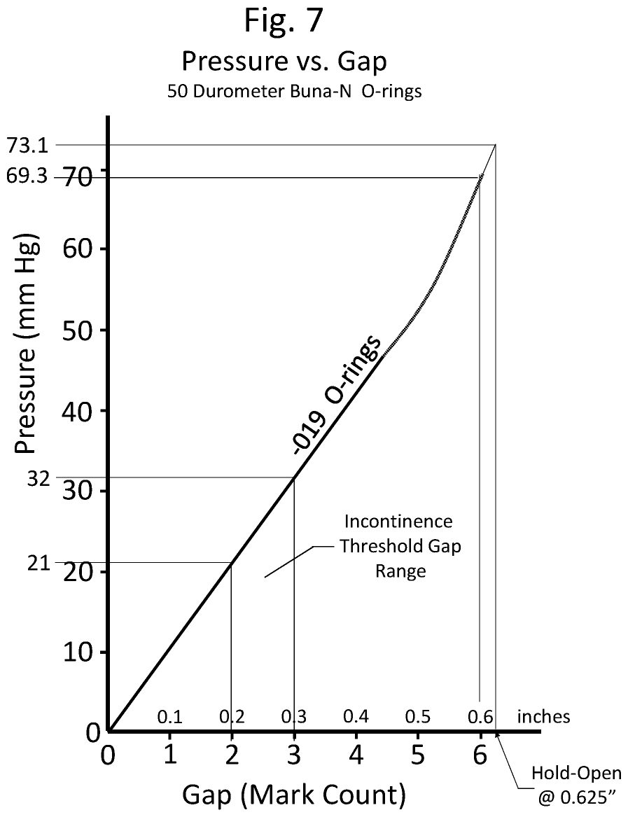

PRESSURE CONTROL—For the reasons previously discussed it is extremely important to control the device pressure applied to the penis. Thus, O-rings 6 are used to control the pressure applied by the pressure disc 15 onto the penis. The pressure on the penis is increased by the pressure disc 15 onto the penis when the adjustment nut 16 is turned clockwise relative to the button 20. The threaded rod 17, passing through the adjustment nut 16, moves the pressure disc 15 toward the penis. The downward force on the pressure disc 15 is created by stretching the O-rings 6. The resisting force of the O-rings 6 is proportional to the amount of stretch of the O-rings 6. FIG. 7 is a graph of the pressure 32 in mm of Hg generated by the pressure disc 15 as a function of the average of the left gap 27 and right gap 28 for a size −019 50 durometer Buna-N O-rings 6. The pressure 32 is equal to the average force 31 developed by the left and right O-rings 6 divided by the projected area 33 of the pressure disc 15. Based on testing, FIG. 7 shows that a gap marking count range of 2.0 to 3.0 is approximately the threshold gap that is required to eliminate incontinence. Any additional pressure due to the bladder can be compensated for by increasing the gap up to the max gap marking count=6.

FIGS. 3 and 4 show that when the gap is at the nominal threshold pressure count of 2.5, the end of the guide 4 in the upper member 2 is flush with the bottom of the channel 5 in the lower member 3. This is especially valuable to a person who has impaired vision.

FIG. 7 shows that the maximum threshold range pressure applied by the pressure disc 15 is 32 mm of Hg. Furthermore, FIG. 7 shows that the pressure just before the hold-open latch engages at a gap marking count of 6.25 is 73.1 mm Hg. Since normal blood pressure range at the heart level is: 60 to 90 Diastolic and 80 to 120 Systolic, and since the hydrostatic difference due to the change in elevation between the heart and the penis of approximately 12 in.=22 mm of Hg, the blood pressure range at the penis level is: 82 to 112 Diastolic and 102 to 142 Systolic. The result is that the blood pressure at the penis level will always be above the max pressure of 73.1 mm of Hg that the pressure disc 15 can generate at the hold-open position. This means that the UITD 1 pressure on the penis is always well below the blood pressure at the penis level and therefore the UITD 1 can be worn throughout the day. Note: a gap marking count of 6.25 is the max gap that can be achieved because above that the UITD 1 goes into the hold-open mode. The ability to control and indicate the pressure that is being applied to the penis is one of the features that differentiates the UITD 1 from all the other incontinence clamps on the market. All other incontinence clamps neither control nor indicate the pressure that the clamp is exerting on the penis. As a result, all other incontinence clamps can restrict blood flow which is why they often require that the clamp be only worn intermittently for a maximum of approximately 2 to 4 hrs.

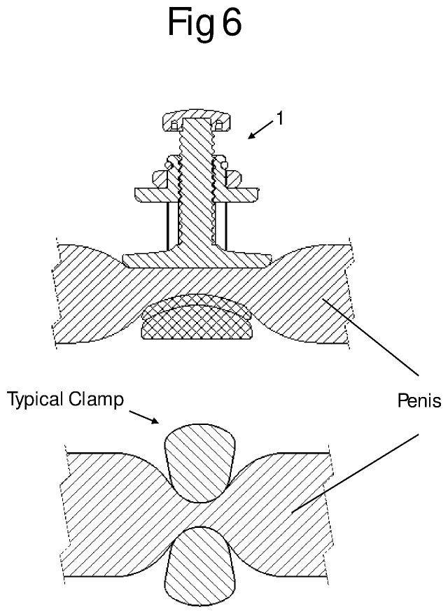

STABILITY—For reasons previously discussed it is important and desirable that the incontinence device be retained on the penis during various daily activities of the user. Stability is defined as the ability to keep the UITD 1 from sliding off the end of the penis. This is an important function because the interior of a flaccid penis has a consistency similar to a piece of liver. That is, while staying connected, the penis material can flow through restrictions such as a clamp allowing the clamp to move along the penis and fall off. FIG. 6 shows that the UITD 1 controls this by the length of the pressure disc 15 engaged with the lower member 3. As the penis is being compressed by the pressure disc 15, the “liver-like” penis material is displaced upstream and downstream of the UITD 1. The amount of material displaced downstream toward the head of the penis along with the length of the engagement of the pressure disc and lower member helps prevent penis material from flowing backward and thus helps prevent the UITD 1 from sliding off the end of the penis. Testing has shown that initially setting the gap between 3 and 4 for several minutes before reducing the gap to the threshold range of 2 to 3 gives the liver-like penis material time to separate into upstream and downstream volumes. The trapped downstream volume helps prevent the UITD 1 from sliding off the end of the penis.

Since it only takes one hand to turn the adjustment nut 16, the UITD 1 can be installed with the UITD 1 button 20 pointing down to minimize the UITD 1 getting caught in clothing during activity. Also, by mounting the UITD 1 with the button 20 pointing down the pressure disc 15 is next to the urethra such that the pressure indicated by the scales is the pressure on the urethra. Stability also requires that the pressure of the pressure disc 15 be applied to the center of the penis. The guides help ensure that the motion between the upper member 2, lower member 3, and threaded rod 17 is in the same plane. The O-rings 6 and O-ring grooves 7 help ensure that the forces are symmetrical. The rigid attachment of the pressure disc 15 to the threaded rod 17 helps maintain that the pressure is applied perpendicular to the threaded rod 17. The curved “V” shape of the lower member helps center the penis in the UITD 1 as pressure is applied.

Thus, the UITD 1 is simple in design and can be readily installed and removed by the user without assistance from others. Furthermore, UITD 1 does not limit the user's mobility and allows the user to lead a normal lifestyle including participating in recreational activities without the discomfort associated with urinary incontinence.

A further embodiment of the present invention is a process for preparing the UITD 1, such as, for example injection molding, vacuum casting, 3D printing and the like. Preferably, the UITD 1 can be prepared using injection molded thermoplastic such as polypropylene that is skin safe and capable of being sterilized in boiling water. Polypropylene also floats if the UITD 1 accidentally dropped into water, such as, while a user is using a toilet.

While preferred embodiments of the present invention have been shown and described in the previous detailed description and drawings it will be obvious to a skilled artisan that such embodiments are exemplary. Without departing from the present invention numerous variations, changes and substitutions will be obvious to a skilled artisan. It should be understood that various alternatives to the embodiments of the present invention described herein may be employed in practicing the present invention. It is intended that the appended claims define the scope of the present invention and that structures and methods within the scope of these claims and their equivalents be covered thereby.

1. A male urinary incontinence threshold device comprising:
an upper member;
a lower member having a curved surface wherein the upper and lower members are dimensioned to enclose a penis of a user;
vertical guides attached to the upper member which allow the upper and lower member to be oriented with respect to each other;
channels located in the lower member which engage the vertical guides and allow the upper and lower members to move up and down vertically parallel to each other;
O-rings which elastically hold the upper and lower members in place;
O-ring groves located in the upper and lower members to hold the O-rings in place;
a catch on the right and left sides of the lower member;
channels in the upper member to allow the catches to slide vertically;
latches in the upper member which engages with the ends of the right and left catches to maintain the upper and lower members in a hold-open position;
diagonal corners located in the upper and lower members which when diagonally squeezed allow the left and right latches to be disengaged from the catches;
a pressure disc;
a threaded rod in which the pressure disc is molded onto the end of the threaded rod;
an adjustment nut which is positioned in and rotates in the upper member and wherein the threaded rod is threaded through;
an O-ring which is stretched over an O-ring groove in the adjustment nut to hold the adjustment nut in place;
a button snapped onto the end of the threaded rod which prevents the button from separating from the threaded rod and helps to pull the upper and lower members apart;
an adjustment nut is turned by means of convolutions around the circumference of the adjustment nut used to turn the adjustment nut relative to the threaded rod;
a scale molded onto both the left and right vertical outside surfaces of the upper member which indicates the distance opening of the left and right gaps, respectively.

2. The device according to claim 1 wherein the scale on each side of the upper member which shows how far the upper and lower members are separated, is equal to how much the O-rings are stretched which can be correlated with the force and pressure being applied to the penis inserted into the device.

3. The device according to claim 1 wherein the distance between the markings on the scales is 0.100 inches.

4. The device according to claim 1 wherein the pressure being applied to an inserted penis is equal to a sum of forces generated by the O-rings used to hold the upper and lower members in place divided by the projected area of the pressure disc where the force is directly proportional to the average of the left and right gaps.

5. The device according to claim 1 that simulates that when gentle pressure is applied to the penis of a user between the thumb and forefinger, urine flow is stopped.

6. The device according to claim 1 formed of a rigid polymer.

7. The device according to claim 6 wherein the rigid polymer is polypropylene.

8. The device according to claim 1 wherein the threaded rod that passes through the nut is constrained to rotate perpendicular to, at the center of, and in the same plane as the upper member.

9. The device according to claim 1 wherein when the nut is turned clockwise relative to the button the pressure disc moves away from to the upper member.

10. The device according to claim 1 wherein the button located at the top end of the threaded rod is used to pull the upper and lower members apart.

11. The device according to claim 1 wherein when the upper and lower members are separated by a predetermined distance, latches located in the upper member engage catches attached to the lower member that slide within the hold-open channel to enable the device to assume a hold-open position and subsequently the latches can be released by alternately applying a diagonal force to the upper right and lower left corners and the upper left and lower right corners of the device.

12. The device according to claim 1 wherein a radiused concave “V” shape in the inside surface of the lower member allows the penis inserted between the lower and upper members to remain in the center of the lower member as pressure is applied.

13. The device according to claim 12 wherein the pressure area of the pressure disc fully engages the penis.

14. The device according to claim 1 wherein the diameter of the pressure disc and depth of the lower member results in a restriction of sufficient length to prevent soft interior penis material from flowing upstream when the device is being pulled off.

15. The device according to claim 1 wherein the end of the threaded rod is molded onto the pressure disc prevents the pressure disc from rotating relative to the threaded rod.

16. A method of controlling urinary incontinence and stopping the flow of urine in a male as needed comprising use of a device according to claim 1.

17. A method according to claim 16 wherein the pressure applied to the penis of a user by the device is always below the blood pressure at the penis.

18. A method according to claim 16 wherein the device may be worn for extended time periods.

19. A method for installing the device according to claim 1 onto the penis by initially installing the device with a gap count of 3 to 4 for several minutes to separate the liver-like interior penis material into upstream and downstream locations for stability, followed by reduction of the gap count to 2 to 3 for comfort.

20. A method for determining that the gap according to claim 1 is at the nominal threshold gap range setting by feeling that the guide 4 in the upper member 2 is flush with the bottom opening of its respective channel 5 in the lower member 3.
